(19) United States
(12) Patent Application Publication
(10) Pub. No.: US 2003/0083739 A1
(43) Pub. Date: May 1, 2003

(54) Rational drug therapy device and methods

(76) Inventor: Robert Cafferata, Santa Rosa, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/254,832

(22) Filed: Sep. 24, 2002

Related U.S. Application Data
(60) Provisional application No. 60/324,846, filed on Sep. 24, 2001.

(51) Int. Cl. A61F 2/06

(52) U.S. Cl. 623/1.42; 604/19; 424/78.05

(57) ABSTRACT

A method and a system for treating vascular in-stent restenosis that combines two disparate drug delivery systems wherein the two drug systems act in a synergistic fashion to produce maximal therapeutic benefit at a targeted site. Controlled delivery of non-toxic, subthreshold doses of a systemic drug is combined with the localized delivery of a second drug via catheter-mediated stent placement to provide therapeutic benefit. Because each drug acts independently via distinct yet related molecular pathways, full therapeutic benefit can be designed as additives and occurs only at the targeted site.

### RELATED APPLICATIONS

The application claims priority of provisional application serial No. 60/324846 filed Sep. 24, 2001, the subject matter of which is hereby incorporated by reference.

### BACKGROUND OF THE INVENTION

Stenosis is the narrowing of a lumen or an opening that occurs in organs, vessels, or other luminal structures within the body. Stenosis is often treated by procedures such as dilation, ablation, atherectomy, or laser treatment. These procedures usually involve the introduction of catheters, guide wires, stents, sheaths, or tubes that are made from synthetic materials. The insertion of these foreign materials, however, leads to certain complications such as luminal scaring and restenosis. Restenosis is attributable to hyperproliferation of vascular smooth muscle, excess epithelialization or stent encrustation. The occurrence of restenosis is dependent upon vessel location, vessel elasticity, lesion length, severity of injury, and an individual's wound healing propensities.

Restenosis is a complication that occurs in thirty to forty percent of all patients that undergo percutaneous transluminal coronary angioplasty (PTCA). Restenosis may be treated by invasive surgical procedures such as coronary artery bypass graft surgery (CABG). However, CABG procedures increase patient suffering, risk of mortality, and associated heath care costs. As a result, less invasive procedures, such as stent implantation, have been developed to treat restenosis.

Stents are mechanical scaffoldings that are inserted into an occluded region of a lumen to provide and maintain patency. Stents are made from a wide variety of materials ranging from metallic materials to biocompatible polymers. In addition to providing luminal patency, stent technology has undergone various improvements. For instance, U.S. Pat. No. 5,102,417, discloses a stent used as a drug delivery vehicle. However, the problem with using a stent as a drug delivery vehicles is that drug delivery may not be sustainable over a long period of time because an effective drug dosage may not be sustainable due to drug dilution, inactivation, degradation, or the like.

Another approach for treating or preventing restenosis has been the administration of various medicaments such as nitric oxide (NO). NO is known to block neointima formation in injured arteries by inhibiting platelet attachment, monocyte infiltration, vascular smooth muscle cell (VSMC) proliferation while activating re-endothelialization and return of vascular homeostasis. In the healthy arteries, endothelial cells secrete NO directly on underlying VSMCs and control VSMC cell number by both a cytostatic (cell cycle blockade) effect and cyclic guanyl monophosphate (cGMP) induced apoptosis. During homeostasis, the mechanism of cGMP induced apoptosis is inactivated by endogenous enzymes, phosphodiesterase, that breakdown VSMC cGMP. That is, apoptosis triggered by NO activation of guanylate cyclase and production of cGMP is blocked. After vascular injury or cardiovascular disease, endothelial cells dysfunction occurs resulting in insufficient NO release. As a result of lower NO concentrations, VSMC relaxation is impaired, and VSMC proliferation and migration is facilitated. Accordingly, treatments using NO has been sought out to prevent or treat restenosis and other complications associated with vascular procedures.

NO treatments, however, have various shortcomings. For example, NO is highly reactive and must be complexed with a “carrier” molecule in order for NO to reach the treatment site. The carrier molecules used to deliver NO to the treatment site are typically small molecules or polymers, but these carrier molecules readily release NO which curtails their ability to deliver NO under physiological conditions. Moreover, the rapid rate of NO release makes it difficult to deliver an effective quantity to the treatment site for extended periods of time or to control the NO dose delivered to the treatment site.

Those carrier molecules known in the art that complex NO may also be cytotoxic. For instance, polymers containing diazeniumdiolate groups have been used to coat medical devices. Decomposition products of these diazeniumdiolate groups may produce nitrosamines, some of which may be carcinogenic. Additionally, NO may react with hemoglobin and can be toxic in individuals with arteriosclerosis.

Furthermore, exogenous NO sources such as pure NO gas are highly toxic, short lived and relatively insoluble in physiological fluids. Consequently, systemic exogenous NO delivery is generally accomplished using organic nitrate prodrugs such as nitroglycerin tablets, intravenous suspensions, sprays and transdermal patches. The human body rapidly converts nitroglycerin into NO; however, enzyme levels and cofactors required to activate the nitrate prodrug are rapidly depleted, resulting in drug tolerance. Moreover, systemic NO administration can have devastating side effects including hypotension and free radical cell damage. Therefore, using organic nitrate prodrugs to maintain systemic anti-restenotic therapeutic blood levels is not currently possible.

Therefore, there is a need to provide for a method for preventing and effectively treating restenosis.

Accordingly, it is an object of the present invention an effective drug delivery system and methods to treat restenosis.

It is yet another object of the present invention to provide an effective drug delivery system that provides non-toxic subthreshold doses of at least two drugs that act in syngergistic fashion to produce maximal therapeutic benefit at a targeted site.

### BRIEF SUMMARY OF THE INVENTION

The present invention relates to a system and a method for treating vascular restenosis that combines two disparate drug delivery systems wherein a drug delivery system acts in a synergistic fashion to produce maximal therapeutic benefit at a targeted site. The present invention permits controlled delivery of non-toxic, subthreshold doses of a systemic drug combined with the precise targeting of catheter-mediated stent placement. Since each drug acts independently via distinct yet related molecular pathways, full therapeutic benefit can be designed as additives and occurs only at the targeted site. Furthermore, restenosis treatment can be actively regulated by controlling systemic drug administration rather than attempting to regulate the drug output of the localized implant.

### DETAILED DESCRIPTION OF THE INVENTION

The present invention relates to a system and a method of treating cardiovascular disorders. In particular, the present invention is useful in treating restenosis by providing a synergistic or additive drug delivery system wherein at least two drugs act in combination to provide maximal therapeutic benefit at a targeted site. Synergistic drug delivery is defined as at least two drugs that operate via distinct yet related molecular pathways wherein the drugs act cumulatively at a targeted site. The targeted site is defined as the site of vascular injury or location within the vasculature where a stent has been placed. The targeted site or localized site have synonymous definitions and may be used interchangeably. More particularly, the synergistic drugs of the present invention are directed to treating restentosis by controlling vascular smooth muscle cell (VSMC) growth while activating re-endothelialization.

The present invention provides therapeutic doses of drugs to a site of vascular damage. During homeostasis, the endothelium plays an important role in cardiovascular regulation by producing various factors such as Nitric oxide (NO). NO is formed by the enzyme nitric oxide synthase (NOS) which cleaves NO from the amino acid, arginine. NO is released from endothelium in response to physiological conditions such as hypoxia and mechanical forces such as shear stress. NO is also released due to factors such as acteylcholine, bradykinin, ATP/ADP, and serotonin. Once nitric oxide synthase is activated, NO is produced and diffuses from the endothelium to VSMC. NO mediates VSMC proliferation and causes VSMC relaxation. Once in the VSMC, NO activates guanylate cyclase to increase cGMP concentration within the cell. The increased cGMP concentration causes muscle relaxation by (1) decreasing intracellular Ca⁺² concentrations, and (2) by reducing the number of active crossbridges which are involved in VSMC contractions.

In contrast, diminished NO concentration may be attributable to endothelial dysfunction. Endothelial dysfunction may be the result of the normal aging process, hypertension, hypercholesteremia, or diabetes. Endothelial dysfunction may also be attributed to physical trauma or surgical procedures such as PCTA. As a result of endothelial dysfunction, NO levels are diminished and this condition may be further exacerbated due to superoxide oxygen (O₂₋) production. O₂₋ inactivates NO thereby inhibiting VSMC relaxation, allowing for monocyte adherence, and causing VSMC proliferation and migration, ultimately resulting in an abnormal narrowing of the blood vessel (i.e., stenosis or restenosis).

In particular, the present invention delivers nitric oxide (NO) and phosphodiesterase inhibitors (PDEI) to a targeted site to limit VSMC proliferation while activating re-endothelialization. The present invention provides the critical doses of NO to allow for proper re-endothelialization due to vascular injury. In particular, NO-induced accumulation of cyclic GMP is amplified in the presence of PDEIs. The present invention prevents restenosis by amplifying the effects of NO. In particular, by providing NO-releasing compounds at a localized site, VSMC growth is regulated. Additionally, restenosis is further reduced by inactivating an enzyme inhibitor that prevents cGMP induced apoptosis. That is, a second drug is provided that removes a regulator of programmed cell death. In particular, PDEls are delivered systemically to trigger the apoptosis. Those skilled in the art will appreciate that PDEIs may be systemically administered orally, intravenously, by suppository, or by other means known in the art.

The present invention permits the controlled delivery of non-toxic, subthreshold doses of a systemic drug combined with the precise targeting of catheter-mediated stent placement. Because each drug acts independently via distinct yet related molecular pathways, full therapeutic benefit can be designed as additives and occurs only at the targeted site. Furthermore, restenosis treatment can be actively regulated by controlling systemic drug administration rather than attempting to regulate the drug output of the localized implant.

According to one embodiment of the present invention, two drugs are administered to prevent and treat restenosis by differing drug delivery mechanisms. In particular, NO is delivered to a localized situs via a drug delivery stent and PDEI is systemically delivered.

According to one embodiment of the present invention, NO is delivered to the injured situs by a stent as disclosed by U.S. patent application Ser. No. 09/865,242, filed May 25, 2001, the entire contents which are hereby incorporated by reference. In particular, the stent is a metallic stent having a silanized metallic surface. The silanized surface can be coupled to NO releasing compounds whereby therapeutic amounts of NO are released to a specific site within a mammalian body. It is contemplated that the stent of the present invention may be placed in areas of stenosis within the coronary or peripheral vasculature.

The metallic stent is exemplary of a medical device having a NO releasing compounds attached to the device surface and is not meant to be limiting. It is also contemplated that NO releasing compounds may attached to the surface of medical devices such as, but not limited to, guide wires, catheters, trocar needles, bone anchors, bone screws, protective platings, hip and joint implants, electrical leads, biosensors and probes.

In a broad aspect of the present invention, the NO-releasing groups are bound to nucleophile residues present in the backbone, or as pendent groups attached to molecules and/or polymers covalently linked to a metal surface. The molecules and polymers having the nucleophile residues may be coupled to the metal surface covalently or non-covalently.

In one embodiment of the present invention the NO-releasing functional groups are 1-substituted diazen-1-ium-1,2-diolates (diazeniumdiolates) referred to hereinafter as NONOates having the general formula (1):

RN[N(O)NO]⁻(CH₂)ₓNH₂⁺R′ (1)

These compounds are disclosed and described in U.S. Pat. Nos. 4,954,526, 5,039,705, 5,155,137, 5,212,204, 5,250,550, 5,366,997, 5,405,919, 5,525,357 and 5,650,447 issued to Keefer et al. and in J. A. Hrabie et al, J. Org. Chem. 1993, 58, 1472-1476, all of which have been incorporated herein by reference.

Generally, the NONOates of the present invention can be easily formed according to formula 2:

X⁻+2NO→X—[N(O)N(O)]⁻ (2)

where X is a nucleophile such as, but not limited to, secondary or primary amines. Suitable nucleophile containing compounds such as, but not limited to, polyethylenimine (PEI) are dissolved in non-aqueous solvents and degassed using alternative cycles of inert gas pressurization followed by depressurization under vacuum. Once the solution has been degassed, the nucleophile is exposed to nitric oxide gas under pressure. The solution's pH is maintained as required to assure the resulting diazeniumdiolate salt's stability. NONOates may be formed on solid substrates, or in solution and precipitated therefrom using an appropriate filter matrix.

In the present invention, the NONOates are formed directly on the surface of a metallic medical device to which reactive nucleophiles have been bonded. For the purposes of the present invention, bonded or coupled refers to any means of stably attaching a nucleophile containing compound to a metallic surface including, but not limited to, ionic bonds, covalent bonds, hydrogen bonds, van der Waals' forces, and other intermolecular forces. Moreover, nucleophile-containing compounds physically entrapped within matrices such as interpenetrating polymer networks and polymeric complexes are considered to be within the scope of the present invention.

The diazeniumdiolates (NONOates) of the present invention are formed by reacting the previously processed metallic medical devices (devices provided with nucleophile residues in accordance with the teachings of the present invention) with NO gas under pressure in an anaerobic environment. It is also possible to entrap NO-releasing compounds within polymer matrices formed on the surface, of the metallic medical devices using the teachings of the present invention. For example, all acetonitrile/THF soluble diazeniumdiolates or other NO-releasing compounds known to those of ordinary skill in the art can be entrapped within polyurethane, polyurea and/or other polymeric matrices on the surface of the metallic medical devices of the present invention. For example, and not intended as a limitation, a polyisocyanate, specifically an aromatic polyisocyanate based on toluene diisocyanate dissolved in a polymer/solvent solution, is added to a mixture containing a saturated polyester resin (polyol), at least one non-aqueous solvent, a NO-releasing compound and a suitable isocyanatosilane. The solution is mixed and the metallic medical device is coated with the solution and then dried. Suitable polyisocyanates include, but are not limited to, m-xylylene diisocyanate, m-tetramethylxylxylerie diisocyanate (meta-TMXDI available from Cytec Industries, Inc., Stamford, Conn.) and Desmodur® CB 60N (available from Baeyer Pittsburgh, Pa.). Polyols useful in this invention include, but are not limited to, polyester polyols, polyether polyols, modified polyether polyols, polyester ether polyols, caster oil polyols, and polyacrylate polyols, including Desmophen® 1800, A450, A365 and A160 (available from Baeyer Pittsburgh, Pa.).

In another embodiment of the present invention, a stent may be complexed with various genes. In particular, a gene encoding nitric oxide synthase (NOS) may be delivered to a site of vascular injury via stent placement. According to this embodiment, the gene encoding NOS is expressed which results in the production of endogenous NO. NOS produces NO by cleaving NO from the amino acid, arginine. Those skilled in the art will appreciate that genes encoding NOS may be locally delivered to a site of vascular injury by gene delivery vehicles such as, but not limited to, liposomes, microspheres, and vectors.

In one embodiment of the present invention, PDEI is the second drug that comprises the system of the present invention. PDEI acts upon the second mechanism of endothelial cell control over VSMC. During homeostasis, endothelial cells produce phosphodiesterases which degrade VSMC cyclic guanyl monophosphate (cGMP). By degrading cGMP, phosphodiesterases block the cGMP induced apoptosis (“programmed death”) of VSMC. PDEI acts to inhibit phosphodiesterase function thereby removing the regulator of cGMP induced apoptosis. As a result, restenosis due to endothelial cell injury is prevented because VSMC proliferation is inhibited.

PDEI may be systemically delivered to the mammalian body. Systemic delivery includes, but is not limited to, oral, sublingual, intravenous, intramuscular, intracranial, intraocular, peritoneal, transdermal, vaginal, or rectal administration of a drug. Additionally, systemic delivery includes drug delivery by inhalation, insufflation, and catheterization. In a preferred embodiment, PDEI is orally delivered to a mammalian subject. By orally delivering PDEI, levels of PDEI may be modulated without the need to actively regulate the drug output of the NO-releasing stent of the present invention.

According to alternate embodiments of the present invention, a plurality of drugs may be systemically administered to relieve the effects of oxidative stress. Oxidative stress is attributable to the loss of cellular redox mechanisms. In healthy vascular endothelial cells, numerous mechanisms are present to inactivate oxidative stressors and maintain the redox balance within the cell. However, after vascular trauma or injury, these cellular redox mechanisms are lost and superoxide levels become elevated. As a highly reactive species, superoxide may react to form hydrogen peroxide, peroxynitrite, and hypochlorous acid. The elevated levels of superoxides and other free radicals have been shown to contribute to the progression of athersclerosis and restenosis. In particular, these pathologies may be further exacerbated by VSMC proliferation, platelet activation, macrophage adhesion, vasospams, lipid peroxidation, and neointimal thickening that results from elevated levels of superoxides. Accordingly, the administration of anti-oxidant compounds such as, but not limited to, superoxide dismutase, glutathione peroxidase, vitamin C, vitamin E, and probucol may counteract oxidative stress.

Moreover, these anti-oxidants would have synergistic effect with locally delivered NO. More specifically, when NO-releasing stent is placed at the site of vascular injury, the effectiveness of local NO delivery may be lost due oxidative stress. That is, NO may react with the superoxide forming peroxynitrite. Thus, the administration of superoxide dismutase or other anti-oxidants would neutralize these oxidative free radicals and increase the efficacy of NO.

In yet another embodiment, anti-inflammatory compounds are the second drug that comprises the system of the, present invention. More specifically, nonsteroidal anti-inflammatory drugs (NSAID) such as, but not limited to, sulindac may be systemically delivered to a subject. Studies have shown that sulindac inhibits macrophage related activities that have been associated with restenosis. Furthermore, studies have suggested that sulindac may inhibit VSMC proliferation and neointimal formation.

Those skilled in the art will appreciate that various combinations of locally delivered drugs and systemically delivered drugs may be provided to produce maximal therapeutic benefit at a target site. For instance, a treatment regime may comprise a locally delivered stent that releases NO and includes genes encoding for NOS in combination with the systemic delivery of PDEI. In yet another drug delivery combination, NO may be delivered to a localized site by a drug delivery stent, NOS and superoxide dismutase genes may be delivered by any known gene delivery vehicle, and PDEI, vitamin C, vitamin E, and sulindac may be delivered systemically.

Typically therapeutic substance/polymer solution can be applied to a medical device such as a stent by either spraying the solution onto the medical device or immersing the medical device in the solution. Whether application is by immersion or by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of therapeutic substance to be applied to the medical device. The total thickness of the polymeric coating will range from approximately 1 micron to about 20 microns or greater. In one embodiment of the present invention the therapeutic substance is contained within a base coat, and a top coat is applied over the therapeutic substance-containing base coat to control release of the therapeutic substance into the tissue.

The polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. As used herein, the term “polymer composition” or “polymer solution” refers to one or more biocompatible polymers suitable for coating a medical device. The “polymer composition” or “polymer solution” may comprise a single polymer of co-polymer, a blend of polymers, a blend of co-polymers, a blend of one or more polymers with one or more co-polymers or any combination thereof.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer-to-therapeutic substance ratio will depend on the efficacy of the polymer in securing the therapeutic substance onto the medical device and the rate at which the coating is to release the therapeutic substance to the tissue of the blood vessel. More polymer may be needed if it has relatively poor efficacy in retaining the therapeutic substance on the medical device and more polymer may be needed in order to provide an elution matrix that limits the elution of a very soluble therapeutic composition. A wide ratio of therapeutic substance-to-polymer could therefore be appropriate and could range from about 10:1 to about 1:100.

In one embodiment of the present invention a vascular stent is coated with a therapeutic substance using a two-layer biologically stable polymeric matrix comprised of a base layer and an outer layer. The stent has a generally cylindrical shape and an outer surface, an inner surface, a first open end, a second open end and wherein the outer and inner surfaces are adapted to deliver an anti-restenotic effective amount of at least one therapeutic substance in accordance with the teachings of the present invention. Briefly, a polymer base layer comprising a polymer solution is applied to stent such that the outer surface is coated with polymer. In another embodiment both the inner surface and outer surface of stent are provided with polymer base layers. The therapeutic substance or mixtures thereof is incorporated into the base layer. Next, an outer layer comprising only a polymer, co-polymer or polymer blend is applied to stent's outer layer that has been previous provide with a base layer. In another embodiment both the inner surface and outer surface of the stent are proved with polymer outer layers.

The thickness of the polymer composition outer layer determines the rate at which the therapeutic substance elutes from the base coat by acting as a diffusion barrier. The polymer composition and therapeutic substance solution may be incorporated into or onto a medical device in a number of ways. In one embodiment of the present invention the therapeutic substance/polymer solution is sprayed onto the stent and then allowed to dry. In another embodiment, the solution may be electrically charged to one polarity and the stent electrically changed to the opposite polarity. In this manner, therapeutic substance/polymer solution and stent will be attracted to one another thus reducing waste and providing more control over the coating thickness.

Another aspect of the present invention are pharmaceutical compositions administered to a patient in need thereof that act synergistically or additively with the therapeutic composition administered via the implanted medical device. A pharmaceutical composition according to the present invention comprises: (1) a synergistically or additive effective amount of a therapeutic substance; and (2) a pharmaceutically acceptable carrier. As defined herein, a synergistically or additive effective amount is defined the concentration of therapeutic substance that achieves an anti-restenotic effect, or other desirable clinical result, when used in combination with another therapeutic substance or pharmaceutical composition.

As described herein, in one embodiment the first therapeutic substance or pharmaceutical composition (drug) is administered systemically and a second therapeutic substance or pharmaceutical composition (drug) is administered locally via a medical device such as a vascular stent wherein the first and second drug act either synergistically or additively to achieve a desirable clinical result.

A pharmaceutically acceptable carrier can be chosen from those generally known in the art including, but not limited to, human serum albumin, ion exchangers, alumina, lecithin, buffer substances such as phosphates, glycine, sorbic acid, potassium sorbate, and salts or electrolytes such as potassium sulfate. Other carriers can be used. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solublizers, stabilizers, and preservatives. Pharmaceutical compositions suitable for use in methods according to the present invention can be administered, for example, by intravenous infusion, orally, topically, intraperitoneally, intravesically, intrathecally, transdermally and combinations thereof. Formulations of pharmaceutical compositions suitable for use in methods according to the present invention can be presented in unit-dose or multi-dose sealed containers, in physical forms such as ampoules or vials.

The pharmaceutical compositions of the present invention typically contain from about 0.1 to 99% by weight (such as 1 to 20% or 1 to 10%) of a synergistic or additive therapeutic compound in a pharmaceutically acceptable carrier. Solid formulations of the compositions for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that may be used include acacia, methylcellulose, sodium carboxymethylcellulose, polyvinylpyrrolidone (Povidone™), hydroxypropyl methylcellulose, sucrose, starch, and ethylcellulose. Lubricants that may be used include magnesium stearates, stearic acid, silicone fluid, talc, waxes, oils, and colloidal silica.

Liquid formulations of the compositions for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions may contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble versions of the compounds may be administered by the drip method, whereby a pharmaceutical formulation containing the antifungal agent and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as water-for-injection, 0.9% saline, or 5% glucose solution. A suitable insoluble form of the compound may be prepared and administered as a suspension in an aqueous base or a pharmaceutically acceptable oil base, such as an ester of a long chain fatty acid (e.g. ethyl oleate).

Transdermal and topical formulations typically contain a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

The pharmaceutical compositions of the present invention are be administered to the patient via conventional means such as oral, subcutaneous, intrapulmonary, transmucosal, intraperitoneal, intrauterine, sublingual, intrathecal, intramuscular or transdermal routes using standard methods. In addition, the pharmaceutical formulations can be administered to the patient via injectable depot routes of administration such as by using 1-, 3-, or 6-month depot injectable or biodegradable materials and methods. Regardless of the route of administration, exemplary dosages in accordance with the teachings of the present invention for these composite compounds range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the composite compound is being administered, the response to treatment, the type and quantity of other medications being given to the patient that might interact with the composite compound, either potentiating it or inhibiting it, and other pharmacokinetic considerations such as liver and kidney function. Generally, initial doses will be modified to determine the optimum dosage for treatment of the particular subject.

Furthermore, the composite compounds of the present invention can be combined with pharmaceutically acceptable excipients and carrier materials such as inert solid diluents, aqueous solutions, or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but not limited to, human serum albumin, ion exchangers, dextrose, alumina, lecithin, buffer substances such as phosphate, glycine, sorbic acid, propylene glycol, polyethylene glycol, and salts or electrolytes such as protamine sulfate, sodium chloride, or potassium chloride. Those skilled in the art will appreciate that other carriers also may be used. Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A drug delivery system for delivering drugs to a localized site comprising:
a first drug; and
a second drug, wherein said first drug and said second drug act synergistically at said localized site.

2. The drug delivery system of claim 1 wherein said first drug is a systemically delivered drug.

3. The drug delivery system of claim 2 wherein said systemically delivered drug is administered orally, sublingually, intravenously, intraocularly, intramuscularly, intracranially, peritoneally, transdermally, vaginally, rectally, by insufflation, by inhalation, or by catheterization.

4. The drug delivery system of claim 2 wherein said first drug is selected from the group consisting of a phosphodiesterase inhibitor, a superoxide dismutase, an anti-inflammatory compound, or an anti-oxidant.

5. The drug delivery system of claim 1 wherein said second drug is a locally delivered drug.

6. The drug delivery system of claim 5 wherein said second drug is nitric oxide or a gene encoding nitric oxide synthase.

7. The drug delivery system of claim 5 wherein said second drug is locally delivered by a stent.

8. A drug delivery system for delivering drugs to a localized site comprising:
a systemically delivered drug; and
a locally delivered drug, wherein said systemically delivered drug and said locally delivered drug act synergistically at a site of local delivery.

9. The drug delivery system of claim 8 wherein said systemically delivered drug is administered orally, sublingually, intravenously, intraocularly, intramuscularly, intracranially, peritoneally, transdermally, vaginally, rectally, by insufflation, by inhalation, or by catheterization.

10. The drug delivery system of claim 8, wherein said systemically delivered drug is selected from the group consisting of a phosphodiesterase inhibitor, a superoxide dismutase, an anti-inflammatory compound, or an anti-oxidant.

11. The drug delivery system of claim 10, wherein said locally delivered drug is delivered by a stent.

12. The drug delivery system of claim 11, wherein said locally delivered drug is nitric oxide, a gene encoding nitric oxide synthase, superoxide dismutase, an anti-inflammatory compound, or an anti-oxidant.

13. A method of delivering a drug to an affected site comprising:
delivering a first drug to an affected site; and
administering a second drug, wherein said first drug and said second drug act synergistically at said affected site.

14. The method of claim 13 wherein said first drug is delivered to said affected site by a stent.

15. The method of claim 14 wherein said first drug is nitric oxide or a gene encoding nitric oxide synthase.

16. The method of claim 13 wherein said second drug is administered orally, sublingually, intravenously, intraocularly, intramuscularly, intracranially, peritoneally, transdermally, vaginally, rectally, by insufflation, by inhalation, or by catheterization.

17. The method of claim 13 wherein said second drug is selected from the group consisting of a phosphodiesterase inhibitor, a superoxide dismutase, an anti-inflammatory compound, or an anti-oxidant.

18. A drug delivery system for delivering drugs to a localized site comprising:
a medical device having a metallic surface, said metallic surface having nitric oxide releasably bound thereto; and
a systemically delivered drug.

19. The drug delivery system of 18 wherein said medical device is selected from the group consisting of stents, guide wires, catheters, trocar needles, bone anchors, bone screws, protective platings, hip and joint implants, electrical leads, biosensors and probes.

20. The drug delivery system of 18 wherein said systemically delivered drug is administered orally, sublingually, intravenously, intraocularly, intramuscularly, intracranially, peritoneally, transdermally, vaginally, rectally, by insufflation, by inhalation, or by catheterization.

21. The drug delivery system of 18, wherein said systemically delivered drug is selected from the group consisting of a phosphodiesterase inhibitor, a superoxide dismutase, an anti-inflammatory compound, or an anti-oxidant.
